(19) United States
(12) Patent Application Publication
(10) Pub. No.: US 2026/0066081 A1
(43) Pub. Date: Mar. 5, 2026

(54) ADJUSTMENT OF GLUCOSE CONTROL PARAMETER(S) BASED ON BOLUS MEDICAMENT DELIVERY DATA

(71) Applicant: INSULET CORPORATION, Acton, MA (US)

(72) Inventors: Joon Bok LEE, Acton, MA (US); Yibin ZHENG, Hartland, WI (US); William WHITELEY, Knoxville, TN (US); Jason O'CONNOR, Acton, MA (US)

(21) Appl. No.: 19/317,562

(22) Filed: Sep. 3, 2025

Related U.S. Application Data
(60) Provisional application No. 63/690,613, filed on Sep. 4, 2024.

(51) Int. Cl. G16H 20/17; G16H 10/60; G16H 40/60

(52) U.S. Cl. CPC G16H 20/17; G16H 10/60; G16H 40/60

(57) ABSTRACT

A framework may be provided for automatically converting a history of manual medicament bolus deliveries to a user into modifications of one or more glucose control parameters of a medicament delivery system. This may eliminate the need for the user to manually adjust the glucose control parameter(s); instead the glucose control parameter(s) may be programmatically adjusted automatically. The glucose control parameter(s) may include, for example, a target glucose level, an input base basal delivery rate, cost function coefficients, and/or a constraint on maximum delivery amounts of medicament. The automatic adjustments may help to customize the glucose control parameter(s) to the user and to minimize the need for user interactions with the medicament delivery system to adjust glucose control parameter(s).

### CROSS-REFERENCE TO RELATED APPLICATIONS

This application claims priority to and the benefit of U.S. Provisional Application No. 63/690,613, filed Sep. 4, 2024, the entirety of which is incorporated herein by reference.

### BACKGROUND

Automated insulin delivery (AID) systems typically allow a user to adjust glucose control parameters. Examples of such adjustable glucose control parameters include target glucose level, delivery constraints, baseline basal delivery rates, cost coefficients, and other clinical parameters. In order to adjust such glucose control parameters, the user must manually specify the adjusted glucose control parameter values. Unfortunately, this may be difficult for the user, and the user may not know what values are suitable for such glucose control parameters. As a result, the glucose control parameters may be set at values that are ill-suited for the user. Moreover, the process of adjusting such glucose control parameters may be confusing and frustrating to the user.

### SUMMARY

In accordance with a first inventive facet, a medicament delivery system for delivering medicament to a user may include a non-transitory processor-readable storage medium storing programming instructions and a processor configured to execute the programming instructions. Executing the instructions may cause the processor to determine a number of boluses of medicament that have been delivered to the user over a period and to determine an adjustment factor based on the determined number of boluses. Executing the programming instructions by the processor may further cause at least one glucose control parameter that is used by the medicament delivery system to control delivery of medicament to the user to be adjusted with the adjustment factor by the processor. The adjusting may cause a rate of medicament delivery to the user by the medicament delivery system to change, resulting in enhanced glucose control for the user automatically.

The one or more glucose control parameters may include at least one of a target glucose level value, a weight coefficient for a glucose cost component of a cost function, a basal delivery rate to the user, and/or a constraint that specifies a maximum delivery amount of medicament for a specified timeframe (i.e., a timeframe of about 15 minutes to 4 hours, more specifically a time frame between about 30 minutes to 2 hours). The adjusting of the one or more glucose control parameters may include at least one of decreasing the target glucose level, increasing the weight coefficient for the glucose cost component of the cost function, increasing the basal delivery rate to the user, and/or relaxing the constraint that specified the maximum delivery amount for the specified timeframe. The adjusting may result in an increase in the rate of medicament delivery (the increase in rate may apply to both the bolus and the basal insulin, i.e., increased size of boluses or higher basal rates). The determining of the number of boluses of the medicament that have been delivered to the user over the period may, in some instances, determine only a number of correction boluses of the medicament that have been delivered to the user over the period. In other instances, the determining of the number of boluses of the medicament that have been delivered to the user over the period may determine a number of correction boluses of the medicament that have been delivered to the user over the period and a number of boluses of the medicament that are not purely correction boluses but that include a correction component that have been delivered to the user over the period. The medicament may be one that manages or affects a glucose level of the user. The adjusting of the at least one glucose control parameter may adjust multiple of the glucose control parameters.

In accordance with another inventive facet, a medicament delivery system for delivering medicament to a user may include a non-transitory processor-readable storage medium storing programming instructions and a processor configured to execute the programming instructions. Executing the programming instruction may cause the processor to determine a sum of bolus doses of medicament that have been delivered to the user over a period and determine an adjustment factor based on the determined sum of the bolus doses. Executing the programming instructions also may cause at least one glucose control parameter used by the medicament delivery system to control delivery of medicament to the user to be adjusted with the adjustment factor by the processor. The adjusting may adjust a rate of insulin delivery to the user by the medicament delivery system.

The at least one glucose control parameter may include at least one of a target glucose level value, a weight coefficient for a glucose cost component of a cost function, a basal delivery rate to the user, and/or a constraint that specifies a maximum delivery amount of medicament for a specified timeframe. The adjusting of the at least one glucose control parameter may include at least one of decreasing the target glucose level, increasing the weight coefficient for the glucose cost component of the cost function, increasing the basal delivery rate to the user, and/or relaxing the constraint that specified the maximum delivery amount for the specified timeframe. The adjusting may result in an increase in the rate of medicament delivery. In some instances, the determining of the number of boluses of the medicament that have been delivered to the user over the period may determine only a number of correction boluses of the medicament that have been delivered to the user over the period. In other instances, the determining of the sum of bolus doses of medicament that have been delivered to the user over the period may determine doses of correction boluses of the medicament that have been delivered to the user over the period and may also determine portions of the boluses of the medicament which are not purely correction boluses that are for correcting glucose level of the user that have been delivered to the user over the period. The medicament may be one that manages or affects a glucose level of the user. The adjusting of the at least one glucose control parameter may entail adjusting multiple of the glucose control parameters.

In accordance with an additional inventive facet, a medicament delivery system for delivering medicament to a user may include a non-transitory processor-readable storage medium storing programming instructions and a processor configured to execute the programming instructions. Executing the programming instructions may cause the processor to determine a number of boluses of medicament that have been delivered to the user over a period and to determine an adjustment factor based on the determined number of boluses and upon at least one recent adjustment factor value. The executing of the programming instructions may further cause the processor to adjust at least one glucose control parameter used by the medicament delivery system to control delivery of medicament to the user with the adjustment factor, wherein the adjusting adjusts a rate of insulin delivery to the user by the medicament delivery system.

The adjustment factor may be determined based on multiple recent adjustment factor values. The determining of the adjustment factor may be based in part on whether a most recent adjustment factor value is greater than a second most recent adjustment factor value. The determining of the adjustment factor may include determining the adjustment factor so that it does not exceed a maximum value.

### BRIEF DESCRIPTION OF THE DRAWINGS

FIG. 1 depicts a block diagram of an illustrative medicament delivery system that is suitable for delivering a medicament to a user in accordance with exemplary embodiments.

FIG. 2 depicts a flowchart of illustrative steps that may be performed in exemplary embodiments to automatically adjust glucose control parameters based on manual medicament bolus delivery data.

FIG. 3 depicts a flowchart of illustrative steps that may be performed in exemplary embodiments to determine and use an adjustment factor to adjust a glucose control parameter.

FIG. 4 depicts a flowchart of illustrative steps that may be performed in exemplary embodiments to set an adjustment factor value based on number of medicament bolus deliveries.

FIG. 5 depicts a flowchart of illustrative steps that may be performed in exemplary embodiments to calculate the adjustment factor value based on a number of boluses with a correction component that were delivered.

FIG. 6 depicts a flowchart of illustrative steps that may be performed in exemplary embodiments to calculate the adjustment factor value based on trend of a number of boluses with a correction factor component that were delivered.

FIG. 7 depicts a flowchart of illustrative steps that may be performed in exemplary embodiments to set an adjustment factor value based on an aggregate quantity of medicament bolus doses.

FIG. 8 depicts a flowchart of illustrative steps that may be performed in exemplary embodiments to set an adjustment factor value based on an aggregate correction bolus component dose.

FIG. 9 depicts a flowchart of illustrative steps that may be performed in exemplary embodiments to adjust a target glucose level using the adjustment factor value.

FIG. 10 depicts a flowchart of illustrative steps that may be performed in exemplary embodiments to adjust a maximum delivery constraint using the adjustment factor value.

FIG. 11 depicts a flowchart of illustrative steps that may be performed in exemplary embodiments to adjust a glucose cost coefficient using the adjustment factor value.

FIG. 12 depicts a flowchart of illustrative steps that may be performed in exemplary embodiments to adjust a basal delivery rate using the adjustment factor value.

FIG. 13 depicts a first flowchart of illustrative steps that may be performed in exemplary embodiments to provide an adjustment factor that increases asymptotically to a fixed value based on number of medicament correction bolus deliveries and number of medicament bolus deliveries that include a correction component.

FIG. 14 depicts a second flowchart of illustrative steps that may be performed in exemplary embodiments to provide an adjustment factor that increases asymptotically to a fixed value based on number of medicament correction bolus deliveries and number of medicament bolus deliveries that include a correction component.

### DETAILED DESCRIPTION

Exemplary embodiments may provide a framework for automatically converting a history of medicament bolus deliveries to a user into modifications of one or more glucose control parameters of a medicament delivery system. The term “glucose control parameter” relates to a (variable) parameter whose adjustment affects the determination, timing, or magnitude of medicament delivery in a glucose control system. Specifically, the exemplary embodiments may look at the history of correction medicament bolus deliveries or more broadly, a history of medicament bolus deliveries that include a correction component (this includes correction medicament bolus deliveries). This may eliminate the need for the user to manually adjust the glucose control parameter(s); instead the glucose control parameter(s) may be programmatically adjusted automatically. The glucose control parameter(s) may include, for example, a target glucose level, an input base basal delivery rate, cost function coefficients, and/or a constraint on maximum delivery amounts of medicament. The automatic adjustments may help to customize the glucose control parameter(s) to the user and to minimize the need for user interactions with the medicament delivery system to adjust glucose control parameter(s).

In exemplary embodiments, the glucose control parameter(s) may be adjusted to increase the aggressiveness of the settings based on one or more factors. The aggressiveness as used herein refers to how quickly the control system seeks to bring glucose levels to the target glucose level. For example, an increased aggressiveness may lead the system to deliver more insulin within a given time frame to correct a blood glucose deviation from the setpoint, compared to the same system at a less aggressive setting. The aggressiveness of the adjusted settings may be controlled by one or more of the factors. These factors may include, for instance, the number of medicament bolus deliveries that are delivered to the user over a timeframe. In some exemplary embodiments, the factors may include the number of medicament correction boluses manually delivered to the user (e.g., manually triggered by the user) over the timeframe. In other exemplary embodiments, the factors may include the number of medicament boluses delivered to the user over the timeframe that include a correction component. For instance, a medicament bolus may include a meal component for offsetting a meal and may also include a correction component for bringing the user's glucose level closer to the target glucose level. The system may identify the correction component by different means. For example, the system may identify a bolus request by a user without an accompanying meal announcement as a medicament bolus that is purely a correction bolus/component. In another example, the system may receive a meal announcement and the user may request a bolus larger than the bolus suggested by the system and the system may classify this as a medicament bolus including a correction component, in particular if blood glucose values for a time period preceding the meal announcement have been elevated. In yet another example, the system may autonomously determine a meal component based on a meal announcement or a meal detection, and may additionally determine a correction component based on an elevated blood glucose level for a time period preceding the meal announcement or meal detection, and classify this as a medicament bolus including a correction component. Still further, in some exemplary embodiments, the factors may include the number of medicament meal boluses manually delivered to the user over the timeframe or the total dose of medicament boluses manually delivered to the user over the timeframe. In some exemplary embodiments, the factors may include a trend of medicament bolus deliveries. It should be appreciated that in some exemplary embodiments, multiple ones of the above-identified factors may be used to adjust the aggressiveness of the glucose control parameter(s). Thus, for example, the number of medicament boluses manually delivered to the user over the timeframe and the total dose of the medicament boluses manually delivered to the user over the timeframe may both be considered as factors in adjusting the glucose control parameter(s).

In some exemplary embodiments, the adjustments may increase the aggressiveness of the glucose control parameter(s) as the number of boluses of the specified type(s) that are manually delivered to the user over the timeframe increases. The notion is that the control system is not providing sufficient glucose level control, and thus the user is manually delivering boluses frequently. Since an aim of the medicament delivery device is to decrease the number of such medicament boluses that are manually delivered to the user and another aim is to provide sufficient glucose level control, the aggressiveness of the glucose control parameter(s) is increased.

In some exemplary embodiments, the adjustments may increase the aggressiveness of the glucose control parameter(s) as the total dose of boluses of the specified type(s) that are delivered to user over the timeframe increases. The notion is that the control system is not providing sufficient glucose level control, and thus, the user is manually delivering medicament boluses doses of notable amounts of medicament to compensate. Since the medicament delivery system wishes to provide sufficient glucose level control so that such medicament bolus doses that are manually delivered to the user are minimized, the aggressiveness of the glucose control parameter(s) may be increased.

In exemplary embodiments, an adjustment factor value is calculated based on the one or more factors. The adjustment factor value may then be used in the equations for calculating the glucose control parameter. The adjustment factor value may dictate the aggressiveness of the glucose control parameter(s) as a result. In other words, the adjustment factor may be used to adjust the aggressiveness of the of the control system.

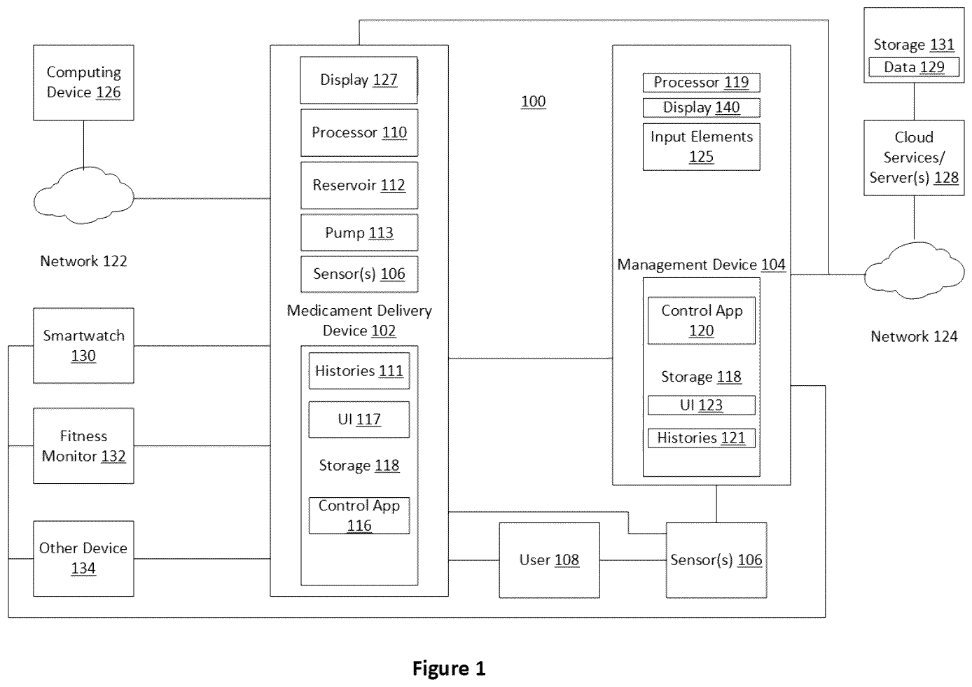

FIG. 1 depicts a block diagram of an illustrative medicament delivery system 100 that is suitable for delivering a medicament to a user 108 in accordance with the exemplary embodiments. The medicament delivery system 100 may include a medicament delivery device 102. The medicament delivery device 102 may be a wearable device that is worn on the body of the user 108 or carried by the user. The medicament delivery device 102 may be directly coupled to the user 108 (e.g., directly attached to a body part and/or skin of the user 108 via an adhesive or the like) with no tubes and an infusion location directly under the medicament delivery device 102, or carried by the user 108 (e.g., on a belt or in a pocket) with the medicament delivery device 102 connected to an infusion site where the medicament is injected using a needle and/or cannula. A surface of the medicament delivery device 102 may include an adhesive to facilitate attachment to the user 108.

The medicament delivery device 102 may include a processor 110. The processor 110 may be, for example, a microprocessor, a logic circuit, a field programmable gate array (FPGA), an application specific integrated circuit (ASIC) or a microcontroller. The processor 110 may maintain a date and time as well as other functions (e.g., calculations or the like). The processor 110 may be operable to execute a control application 116 encoded in computer programming instructions stored in the storage 114 that enables the processor 110 to direct operation of the medicament delivery device 102. The control application 116 may be a single program, multiple programs, modules, libraries or the like. The processor 110 also may execute computer programming instructions stored in the storage 114 for a user interface (UI) 117 that may include one or more display screens shown on display 127. The display 127 may display information to the user 108 and, in some instances, may receive input from the user 108, such as when the display 127 is a touchscreen.

The control application 116 may control delivery of the medicament to the user 108 per a control approach like that described herein. The control application may use a glucose prediction model as described below for predicting future glucose levels of the user 108. The storage 114 may hold histories 111 for a user, such as a history of basal medicament deliveries, a history of bolus medicament deliveries, and/or other histories, such as a meal event history, exercise event history, glucose level history, other analyte level history, and/or the like. In addition, the processor 110 may be operable to receive data or information. The storage 114 may include both primary memory and secondary memory. The storage 114 may include random access memory (RAM), read only memory (ROM), optical storage, magnetic storage, removable storage media, solid state storage or the like.

The medicament delivery device 102 may include a tray or cradle and/or one or more housings for housing its various components including a pump 113, a power source (not shown), and a reservoir 112 for storing medicament for delivery to the user 108. In some embodiments, a structure, such as part of the housing, may be provided for holding a vial or other source of medicament rather than including a reservoir 112. A fluid path to the user 108 may be provided, and the medicament delivery device 102 may expel the medicament from the reservoir 112 or other medicament source to deliver the medicament to the user 108 using the pump 113 via the fluid path. The fluid path may, for example, include tubing coupling the medicament delivery device 102 to the user 108 (e.g., tubing coupling a cannula to the reservoir 112), and may include a conduit to a separate infusion site. The medicament delivery device 102 may have operational cycles, such as every 5 minutes, in which basal doses of medicament are calculated and delivered as needed. These steps are repeated for each cycle.

There may be one or more communications links with one or more devices physically separated from the medicament delivery device 102 including, for example, a management device 104 of the user 108 and/or a caregiver of the user 108, sensor(s) 106, a smartwatch 130, a fitness monitor 132 and/or another variety of device 134. The communication links may include any wired or wireless communication links operating according to any known communications protocol or standard, such as Bluetooth®, Wi-Fi, a near-field communication standard, a cellular standard, or any other wireless protocol.

The medicament delivery device 102 may interface with a network 122 via a wired or wireless communications link. The network 122 may include a local area network (LAN), a wide area network (WAN), a cellular network, a Wi-Fi network, a near field communication network, or a combination thereof. A computing device 126 may be interfaced with the network 122, and the computing device may communicate with the medicament delivery device 102.

The medicament delivery system 100 may include one or more sensor(s) 106 for sensing the levels of one or more analytes. The sensor(s) 106 may be coupled to the user 108 by, for example, adhesive or the like and may provide information or data on one or more medical conditions, physical attributes, or analyte levels of the user 108. The sensor(s) 106 may be physically separate from the medicament delivery device 102 or may be an integrated component thereof. The sensor(s) 106 may include, for example, glucose monitors, such as continuous glucose monitors (CGM's) and/or non-invasive glucose monitors. The sensor(s) 106 may include ketone sensors, other analyte sensors, heart rate monitors, breathing rate monitors, motion sensors, temperature sensors, perspiration sensors, blood pressure sensors, alcohol sensors, or the like. Some sensors 106 may also detect characteristics of components of the medicament delivery device 102. For instance, the sensors 106 in the medicament delivery device may include voltage sensors, current sensors, temperature sensors and the like.

The medicament delivery system 100 may or may not also include a management device 104. In some embodiments, no management device is needed as the medicament delivery device 102 may manage itself. The management device 104 may be a special purpose device, such as a dedicated personal diabetes manager (PDM) device. The management device 104 may be a programmed general-purpose device, such as any portable electronic device including, for example, a dedicated controller, such as a processor, a micro-controller, or the like. The management device 104 may be used to program or adjust operation of the medicament delivery device 102 and/or the sensor(s) 106. The management device 104 may be any portable electronic device including, for example, a dedicated device, a smartphone, a smartwatch, or a tablet. In the depicted example, the management device 104 may include a processor 119 and a storage 118. The processor 119 may execute processes to manage a user's glucose levels and to control the delivery of the medicament to the user 108. The medicament delivery device 102 may provide data from the sensors 106 and other data to the management device 104. The data may be stored in the storage 118. The processor 119 may also be operable to execute programming code stored in the storage 118. For example, the storage 118 may be operable to store one or more control applications 120 for execution by the processor 119. Storage 118 may also be operable to store historical information such as medicament delivery information, analyte level information, user input information, output information, or other historical information. The control application 120 may be responsible for controlling the medicament delivery device 102, such as by controlling the automated medicament delivery (AMD) (or, for example, automated insulin delivery (AID)) of medicament to the user 108. The storage 118 may store the control application 120, histories 121 like those described above for the medicament delivery device 102, and other data and/or programs.

A display 140, such as a touchscreen, may be provided for displaying information. The display 140 may display user interface (UI) 123. The display 140 also may be used to receive input, such as when the display is a touchscreen. The management device 104 may further include input elements 125, such as a keyboard, button, knobs, or the like, for receiving input of the user 108.

The management device 104 may interface with a network 124, such as a LAN or WAN or combination of such networks, via wired or wireless communication links. The management device 104 may communicate over network 124 with one or more servers or cloud services 128. Data, such as sensor values, may be sent, in some embodiments, for storage and processing from the medicament delivery device 102 directly to the cloud services/server(s) 128 or instead from the management device 104 to the cloud services/server(s) 128.

Other devices, like smartwatch 130, fitness monitor 132 and device 134 may be part of the medicament delivery system 100. These devices 130, 132 and 134 may communicate with the medicament delivery device 102 and/or management device 104 to receive information and/or issue commands to the medicament delivery device 102. These devices 130, 132 and 134 may execute computer programming instructions to perform some of the control functions otherwise performed by processor 110 or processor 119, such as via control applications 116 and 120. These devices 130, 132 and 134 may include displays for displaying information. The displays may show a user interface for providing input by the user 108, such as to request a change or pause in dosage, or to request, initiate, or confirm delivery of a bolus of medicament, or for displaying output, such as a change in dosage (e.g., of a basal delivery amount) as determined by processor 110 or management device 104. These devices 130, 132 and 134 may also have wireless communication connections with the sensor 106 to directly receive analyte measurement data.

The functionality described herein for the exemplary embodiments may be under the control of or performed by the control application 116 of the medicament delivery device 102 or the control application 120 of the management device 104. In some embodiments, the functionality wholly or partially may be under the control of or performed by the cloud services/servers 128, the computing device 126 or by the other enumerated devices, including smartwatch 130, fitness monitor 132 or another wearable device 134.

In the closed loop mode, the control application 116, 120 determines the medicament delivery amount for the user 108 on an ongoing basis based on a feedback loop. For a medicament delivery device that uses insulin, for example, the aim of the closed loop mode is to have the user's glucose level at a target glucose level or within a target glucose range.

In some embodiments, the medicament delivery device 102 need not deliver one medicament alone. Instead, the medicament delivery device 102 may deliver a first medicament, such as insulin, for lowering glucose levels of the user 108 and also deliver a second medicament, such as glucagon, for raising glucose levels of the user 108. The medicament delivery device 102 may deliver a glucagon-like peptide (GLP)-1 receptor agonist medicament for lowering glucose or slowing gastric emptying, thereby delaying spikes in glucose after a meal. The medicament delivery device 102 may deliver a gastric inhibitory polypeptide (GIP) or a dual GIP-GLP receptor agonist. In other embodiments, the medicament delivery device 102 may deliver pramlintide, or other medicaments that may substitute for insulin. More generally, the medicament delivery device 102 may deliver a medicament for managing and/or affecting glucose levels of the user 108. In other embodiments, the medicament delivery device 102 may deliver concentrated insulin. In some embodiments, the medicament or medicament delivered by the medicament delivery device may be a coformulation of two or more of those medicaments identified above. In an exemplary embodiment, the medicament delivery device delivers insulin; accordingly, reference will be made throughout this application to insulin and an insulin delivery device, but one of ordinary skill in the art would understand that medicaments other than insulin can be delivered in lieu of or in addition to insulin.

Insulin deliveries to the user 108 may be bolus insulin deliveries or basal insulin deliveries. Bolus insulin deliveries tend to be to offset the expected rise in glucose level of the user 108 from ingesting a meal or for correcting a persistently elevated glucose level (i.e., one that is persistently higher than a target glucose level). Boluses tend to be one time deliveries for offsetting a meal or for correcting a glucose level and tend to be larger than basal insulin deliveries. Insulin boluses may be delivered manually by the user 108, such as via a syringe, or may, in some exemplary embodiments, be delivered by the medicament delivery device 102. Basal insulin doses tend to be smaller than insulin bolus doses and are delivered periodically, such as once each operational cycle of the control approach of the medicament delivery device 102 (e.g., every 5 minutes). The aim of the basal insulin deliveries is to keep the user's glucose level within a target range that is desirable using small ongoing insulin doses.

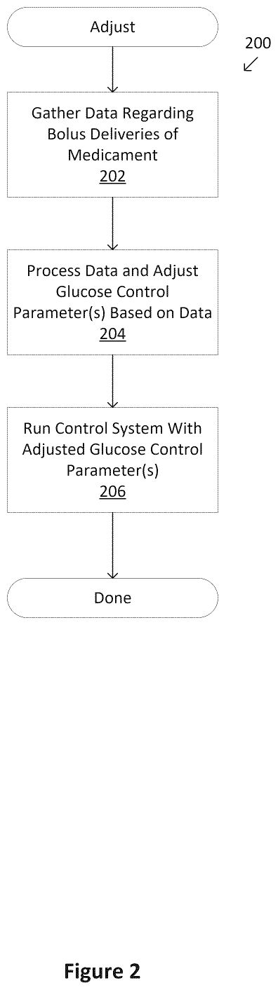

FIG. 2 depicts a flowchart 200 of illustrative steps that may be performed in exemplary embodiments to adjust one or more glucose control parameters, such as to increase the aggressiveness of the system in order to decrease correction bolus activity. At 202, data is gathered by the control application 116 or 120 regarding bolus deliveries of medicament. These boluses may have been delivered manually or by the medicament delivery system 100. The user may input information regarding requested manual medicament bolus deliveries via a user interface provided on the management device 104 or the medicament delivery device 102. In some exemplary embodiments, other devices, like the smartwatch 130, the fitness monitor 132, or another type of device 134, may provide the user interface for entering the information regarding the bolus deliveries of medicament. The information may include information such as the time, the dose, the type of medicament, and whether the bolus is extended or not. The information may be passed to the management device 104 and/or medicament delivery device 102. In other embodiments, the information regarding automated medicament bolus deliveries is generated and stored by the control application 116 or 120.

At 204, the data regarding bolus deliveries of medicament is processed to adjust one or more glucose control parameters. The one or more glucose control parameters may be adjusted to decrease the number of medicament bolus deliveries used and/or to decrease the amount of medicament delivered as bolus deliveries. Examples of how such adjustments may be performed are described below. At 206, the control system provided by the control application 116 or 120 then may be run with the one or more adjusted glucose control parameters.

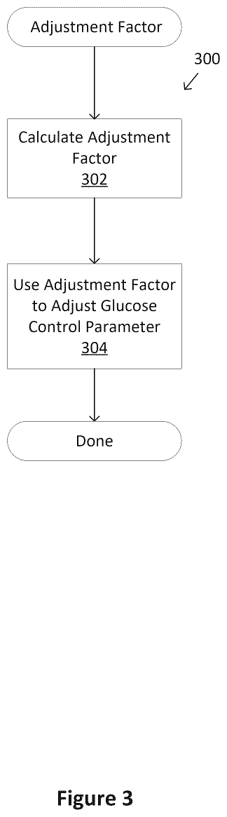

FIG. 3 depicts a flowchart 300 of illustrative steps that may be performed in exemplary embodiments to adjust one or more glucose control parameters responsive to medicament bolus delivery data. At 302, the medicament bolus delivery data may be used to calculate an adjustment factor. The adjustment factor may be a value that is calculated as described below. At 304, the adjustment factor may be used to adjust one or more glucose control parameters. There may be formulas that are particular to each glucose control parameter that is adjusted to specify how to apply the adjustment factor. To reduce the number and quantity of medicament bolus deliveries that are provided for correction purposes, the adjustment or adjustments may increase the aggressiveness of the control approach.

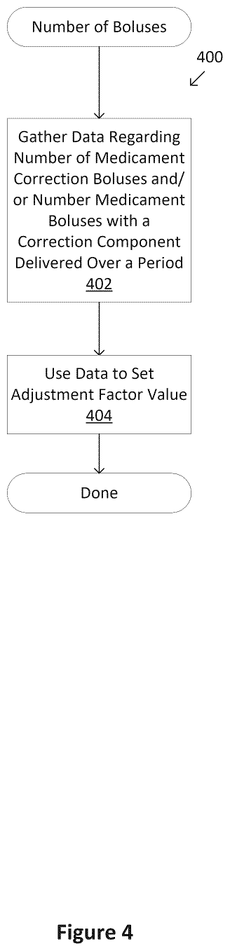

The relevant medicament bolus delivery data for calculating the adjustment factor (see 302) may be, for example, data regarding how many boluses of medicament are delivered over a reference time frame. FIG. 4 depicts a flowchart 400 of illustrative steps that may be performed when the number of boluses is used to determine the adjustment factor. At 402, data regarding the number of correction medicament boluses or a number of medicament boluses with a correction component that are delivered over a period may be determined. For example, the period may be a single day or another number of days, such as three days, or one week or another number of weeks. In some embodiments the period is between 12 hours to 2 weeks, more specifically between about 1 day and 7 days, and in particular between about 2 days and 5 days. At 404, the data may be used to set the value of the adjustment factor.

One example of how the data regarding the number of medicament boluses delivered in a period may be used to set a value for the adjustment factor (see 404) is captured by the following equation:

$${P}_{a d j} ( k ) = 1 + min ( \frac{{Σ}_{j = 1}^{288} {N}_{c o r r} ( k - j )}{20} , 0.4 ) \qquad ( Equation l )$$

$$where$$

$${N}_{c o r r} ( j ) = \{ \begin{matrix}1 & {l}_{c o r r} ( j ) > 0 \\ 0 & {l}_{c o r r} ( j ) = 0\end{matrix} \qquad ( Equation 2 )$$

P_{adj}(k) is the adjustment factor for operational cycle k (which are presumed to be 5 minutes in length so that there are 288 5-minutes cycles in a 24 hour period), min( ) is a function that chooses a minimum value among the inputs, I_{corr}(j) is a function that counts the number of boluses with a correction component or that are correction boluses in cycle j, N_{corr}(j) indicates where there is at least one medicament bolus that is a correction bolus or with a correction component as counted by the I_{corr}(j) function. It should be appreciated that in some embodiments only correction boluses may be counted by I_{corr}(j), whereas in other embodiments correction boluses and boluses that have a correction component (e.g., a meal bolus that has a meal/carb component and a correction component) may both be counted or only medicament boluses with a correction component are counted. An example of a bolus with a correction component is a bolus that includes a meal component that is aimed at offsetting the insulin rise due to ingestion of carbohydrates and an additional amount of medicament that decreased the glucose level of the user 108.

Equation 1 is only one example, but it sums the number of cycles that include a correction medicament bolus delivery or that include a medicament bolus delivery with a correction component (depending on the embodiment) and divides the resulting sum by the exemplary value of 20. It should be appreciated that 20 is a tunable value. The value 20 may be chosen in one embodiment because if the sum of correction boluses (or cycles in which a correction bolus is delivered) is greater than 8, the exemplary value of 0.4 (which is also a tunable value) will be chosen by the min( ) function rather than the ratio, and 8/20 is 0.4. The value 8 represents an empirically-derived average number of cycles where a user delivers a medicament bolus with a correction component. Equation 2 indicates that N_{corr}(j) may be set at zero if there are no medicament bolus deliveries with a correction component or no correction medicament bolus deliveries in cycle j. Otherwise, it may have a value of 1.

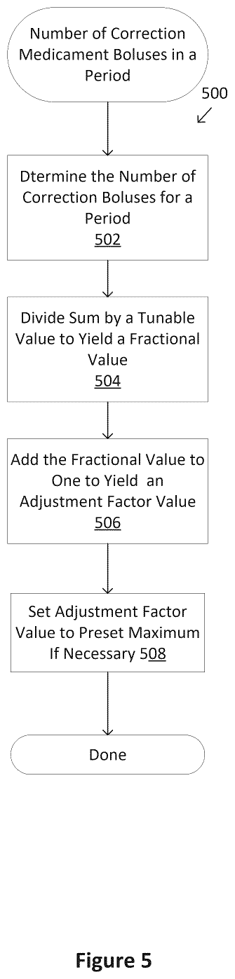

FIG. 5 depicts a flowchart 500 of illustrative steps that may be performed in exemplary embodiments when adopting an approach that uses exemplary equations 1 and 2 or similar equations in calculating the adjustment factor. In this instance, the bolus deliveries being counted are limited to correction boluses. At 502, the number of correction boluses delivered for a period may be determined. The actual number of boluses delivered may be counted, or instead, the number of cycles have at least one bolus delivered during the cycles may be counted. For this instance, I_{corr}(j) refers to the number of correction boluses during cycle k and does not include the number of other types of boluses, such as meal boluses, that include a correction component. In exemplary equation 1, the sum is calculated as

$${Σ}_{j = 1}^{288} {N}_{c o r r} ( k - j ) .$$

At 504, the sum may be divided by a tunable value, such as 20 in this example, to yield a fractional value. At 506, the fractional value may be added to 1 to yield the adjustment value. Thus, the adjustment may be equal to

$$1 + \frac{{Σ}_{j = 1}^{288} {N}_{c o r r} ( k - j )}{20} .$$

In some embodiments, at 508, the adjustment factor may be adjusted to a maximum, such as 1.4, if it would otherwise exceed the maximum. The maximum is realized by the min( ) function selecting the smaller of

$$1 + \frac{{Σ}_{j = 1}^{288} {N}_{c o r r} ( k - j )}{20}$$

and 0.4 in exemplary equation 1. The selected minimum may be added to 1 in exemplary equation 1. It should be appreciated that exemplary equation 2 need not be used in which case N_{corr}(k−j) may be replaced with I_{corr}(k−j) in equation 1.

In other exemplary embodiments, the trend of the number of medicament bolus deliveries with a correction component in a period is reviewed. If the average number of medicament boluses delivered has increased over a period, then adjustments may be made to one or more glucose control parameters. A suitable illustrative and exemplary equation for that instance is:

$${P}_{adj} ( k ) = min ( 1 + 0.05 \cdot max ( 0 , {\sum }_{j = 1}^{288} {N}_{c o r r} ( k - j ) - {\sum }_{j = 289}^{576} {N}_{c o r r} ( k - j ) ) , 1 \text{.4} ) . \qquad ( Equation 3 )$$

However, other equations may be used instead.

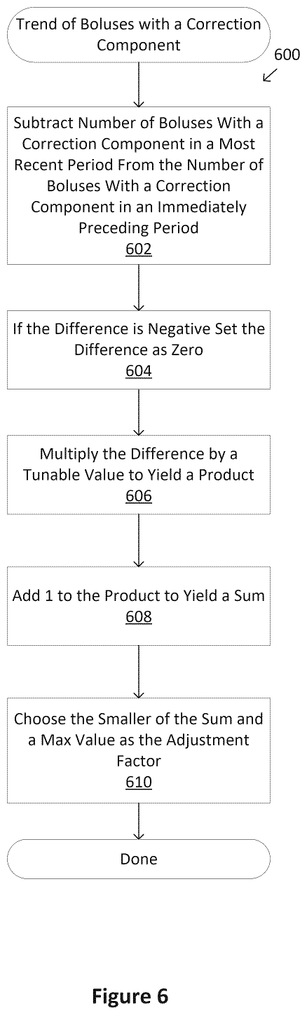

FIG. 6 depicts a flowchart 600 of illustrative steps that may be performed in exemplary embodiments in which the trend of boluses may drive adjustments to one or more glucose control parameters. At 602, a difference between the number of medicament boluses with a correction component that were delivered in a most recent period and the number of medicament boluses with a correction component that were delivered in an immediately preceding period may be determined. For example, suppose 10 medicament boluses with a correction component were delivered in the most recent period and 7 were delivered in the immediately preceding period. In such an example the difference would be +3. In equation 3, the difference is captured by

$${Σ}_{j = I}^{288} {N}_{c o r r} ( k - j ) - {Σ}_{j = 289}^{576} {N}_{c o r r} ( k - j ) ,$$

where j is a cycle index, the cycles are 5 minutes in length, and the equation calculates the difference between two successive 24 hour periods. At 604, if the difference is negative, the difference is set to 0 to prevent negative values from being used in the calculations for the adjustment factor. At 606, the difference may be multiplied by a tunable value. In exemplary equation 3, the exemplary tunable value is 0.05, but other values may be chosen. The tunable value modulates the magnitude of the adjustment. At 608, 1 may be added to the product. The resulting sum is captured as

$$1 + 0.05 \cdot max ( 0 , {\sum }_{j = 1}^{288} {N}_{c o r r} ( k - j ) - {\sum }_{j = 289}^{576} {N}_{c o r r} ( k - j ) )$$

in equation 3. At 610, the smaller of the maximum value (e.g., 1.4) and the sum may then be chosen as the value of the adjustment factor P_{adj}(k).

It should be appreciated that a maximum value need not be used. Moreover, the time period need not be 24 hours bur rather may be of a different duration, such as a period of several hours, multiple days, weeks, etc., i.e., successive 48 hour or 168 hour periods. Further, other equations may be used to calculate the adjustment factor. The adjustment factor may be adjusted based on the trend in other manners.

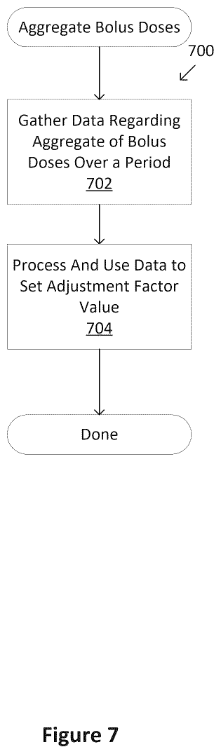

As mentioned above, the setting of the adjustment factor value may also be based on the aggregate quantity of medicament bolus doses delivered to the user 108 over a reference period. FIG. 7 depicts a flowchart 700 of steps that may be performed in exemplary embodiments in this regard. At 702, data regarding the aggregate of medicament bolus doses delivered over a period may be gathered. The data may include, for instance, dose amount, time and date of delivery of the dose, and medicament type. For example, the data may specify that an insulin bolus was delivered at 8 pm on Oct. 1, 2024 and may also specify that the insulin was fast-acting insulin. At 704, the data may be processed and used to set a value for the adjustment factor.

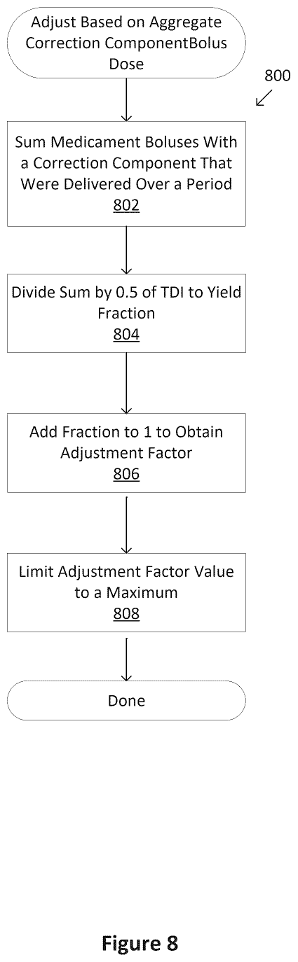

An exemplary equation for calculating the adjustment factor from the quantity of medicament bolus deliveries for boluses with a correction component is:

$${P}_{adj} ( k ) = min ( 1 + \frac{{Σ}_{j = 1}^{288} {I}_{c o r r} ( k - j )}{0.5 \cdot TDI} , 1.4 ) \qquad ( Equation 4 )$$

where TDI refers to total daily insulin for the user 108. FIG. 8 depicts a flowchart 800 of illustrative steps that may be performed to calculate the adjustment factor using an equation, such as equation 4. At 802, the medicament boluses with a correction component that were delivered over a period may be summed. The period may be, for example, several hours, a day, multiple days, weeks, etc. In some exemplary embodiments, the medicament bolus deliveries that are summed may be only those that are solely for correction purposes, and in other embodiments, the medicament bolus deliveries that are summed may include medicament bolus deliveries where only portions are for correction purposes as well. In exemplary embodiments, the number of instances of boluses for correction purposes are summed. At 804, the sum may be divided by one half of the TDI for the user 108 to yield a fraction. One half of TDI may correspond to an ideal amount for medicament bolus deliveries per day, the other half representing an ideal amount of medicament basal deliveries per day. Hence, the fraction represents the ratio of the actual medicament correction bolus deliveries to the ideal daily amount for all medicament bolus deliveries in a day. At 806, the fraction may be added to 1 to set the adjustment factor value. In some instances, an exemplary maximum value may be used. In those instances, at 808, the smaller of the sum and the exemplary value of 1.4 may be used as the adjustment factor. As mentioned before, no maximum need be used. Further, maximum values other 1.4 may be used. Still further, other formulas may be used to determine the adjustment factor from aggregate bolus deliveries.

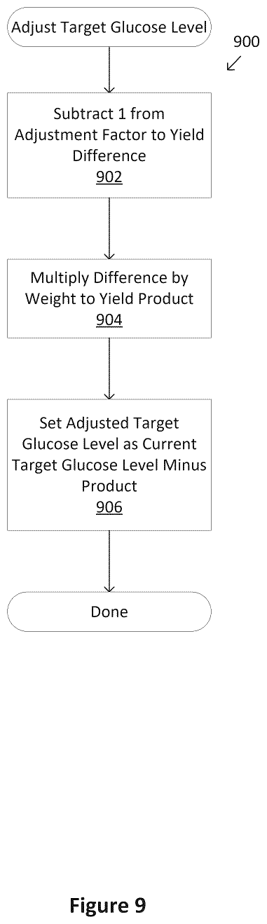

One or more glucose control parameters may be adjusted using the adjustment factor (see 304). A first exemplary glucose control parameter that may be adjusted is a target glucose concentration level for the user 108. An illustrative formula for adjusting the target glucose level of the user 108 is:

$$S {P}^{′} ( k ) = S P ( k ) - 10 ( {P}_{adj} ( k ) - 1 ) \qquad ( Equation 5 )$$

where SP(k) is the current set point (i.e., glucose level target) and SP′(k) is the adjusted set point that has been adjusted using the adjustment factor. It should be appreciated that the value 10 is a tunable value and other values may be used. FIG. 9 depicts a flowchart 900 of illustrative steps that may be performed to adjust the target glucose level, such as by using exemplary equation 5. At 902, one may be subtracted from the adjustment factor value (i.e., P_{adj}(k)−1) to yield a difference. At 904, the difference may be multiplied by a tunable weight value, such as 10 in this example. The resulting difference represents the magnitude of the adjustment to be made to the target glucose level. At 906, the difference may be subtracted from the target glucose level. Reducing the glucose level target makes the control system more aggressive in driving the glucose level of the user to a lower level.

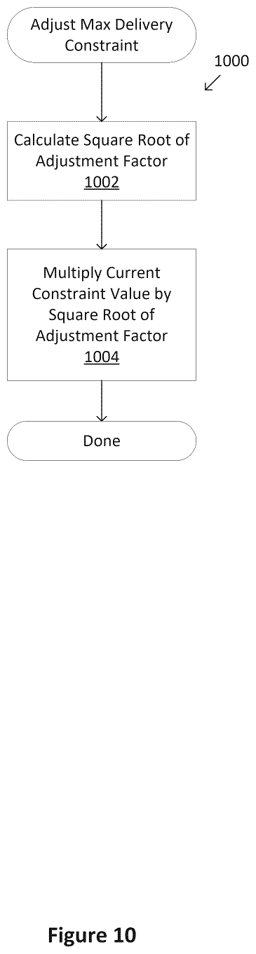

Another glucose control parameter that may be adjusted using the adjustment factor is a maximum delivery constraint. This constraint constrains the amount of medicament that can be delivered over a period, such as per cycle or per hour. FIG. 10 depicts a flowchart 1000 of illustrative steps that may be performed in exemplary embodiments to adjust the maximum delivery constraint. At 1002, a square root of the adjustment factor may be determined. Thus, for example if the adjustment factor is 1.2, the square root value of approximately 1.095 is calculated. At 1004, the constraint amount may be multiplied by the square root to produce the adjusted constraint. Increasing the constraint enables the control system to be more aggressive in attempting to reduce or eliminate positive glucose excursions (i.e., instances where the glucose level of the user 108 is above the target glucose level for a period). This example may be expressed as:

$${U}_{max}^{′} ( k ) = {P}_{adj}^{0.5} {U}_{max} ( k ) \qquad ( Equation 6 )$$

where Uₘₐₓ(k) is the current maximum amount for the period at cycle k and Uₘₐₓ(k) is the adapted maximum amount.

An additional glucose level control parameter that may be adjusted by the adjustment factor is to adjust a glucose cost coefficient Q in a cost function used by the control application 116 or 120 that is used in determining best basal doses on an ongoing basis, such as for each cycle. An example of a cost function is:

$$J ( {I}_{r e c} ) = {Q ( f ( {I}_{r e c} ) - {G}_{target} )}^{n} + {R ( {I}_{r e c} - {I}_{b} )}^{m} \qquad ( Equation 7 a )$$

where J is the total cost, I_{rec} is the current recommended insulin delivery being assessed for the total cost, Q is the glucose cost coefficient, ƒ(I_{rec}) is any generic function to associate this recommended insulin delivery with a corresponding expected glucose value, G_{target} is the current control target, R is the insulin cost coefficient, I_{b} is the current baseline insulin delivery, and n and m are generic coefficients representing any scaling of the penalties for glucose and/or insulin excursions. In more general terms, the glucose level control parameter may be used to adjust a cost function which is a mathematical expression that assigns a total cost to a candidate insulin delivery based on predicted glucose deviation and insulin deviation. The insulin dose that minimizes this cost may be selected as the recommended or to be administered dose.

The terms (ƒ(I_{rec})−G_{target}) may be viewed as glucose cost of delivering the recommended dose of insulin. The function ƒ(I_{rec}) is a function that associates the recommended insulin delivery dosage with a corresponding expected glucose level of the user. Thus, there is a penalty for the glucose level not being at the target level. The terms (I_{rec}−I_{b}) may be viewed as the insulin cost of delivering the recommended dose of insulin. There is a penalty for the insulin delivery dosage varying from the basal dosage. Q is a glucose cost weight coefficient for weighing the glucose cost, and R is an insulin cost weight coefficient for weighing the insulin cost.

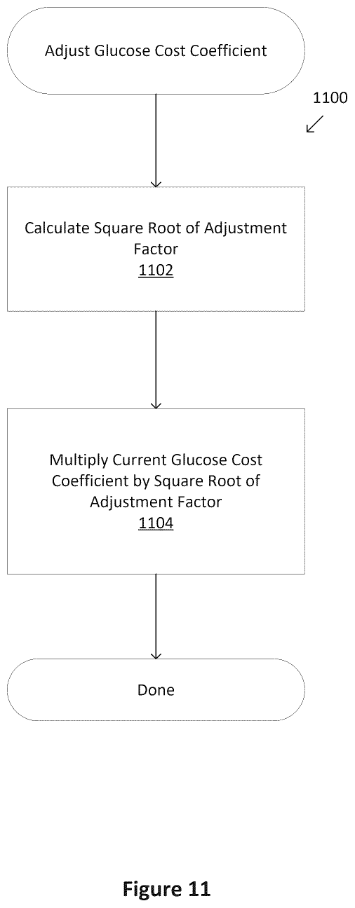

FIG. 11 depicts a flowchart 1100 of illustrative steps that may be performed in exemplary embodiments to adjust the glucose cost coefficient using the adjustment factor. At 1102, the square of the adjustment factor may be calculated. At 1104, the current glucose cost coefficient may be multiplied by the square of the adjustment factor to produce the adapted glucose cost coefficient. Increasing the glucose cost coefficient makes the system more aggressive in attempting to reduce or eliminate positive glucose excursions. Hence, the exemplary adapted glucose cost coefficient may be expressed as:

$${Q}^{′} ( k ) = {P}_{adj}^{2} Q ( k ) \qquad ( Equation 7 b )$$

where Q(k) is the current glucose cost coefficient value and Q′(k) is the adjusted glucose cost coefficient value. It should be appreciated that the adjusted glucose coefficient may be calculated in other ways. For example, instead of the square of the adjustment factor P_{adj} being used, a weight that is a multiple, like 1.1 or 1.2 of the adjustment factor could be used in the equation to determine the adjusted glucose cost coefficient Q′(k).

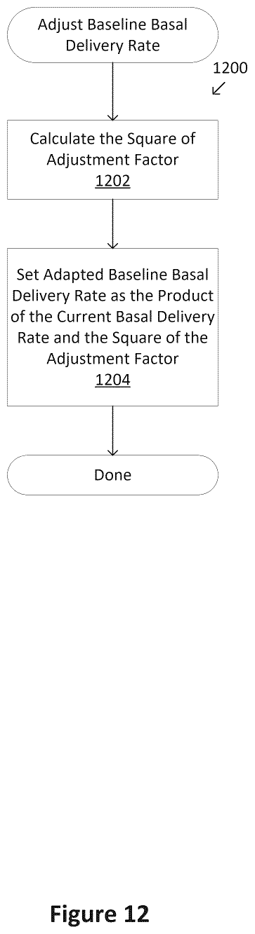

The baseline basal delivery rate is a further glucose level control parameter that may be adjusted using the adjustment factor. The baseline basal rate is a rate provided to the control system and may be used in the cost function (equation 7a) as I_{b}. FIG. 12 depicts a flowchart 1200 of illustrative steps that may be performed in exemplary embodiments to adjust the baseline basal delivery rate. At 1202, the square root of the adjustment factor may be determined. A different numerical operation may be performed other than a square root operation. At 1204, the adjusted baseline basal delivery rate may be set as the product of the square root of the adjustment factor and the current basal delivery rate. Increasing the baseline basal delivery rate increases the aggressiveness of the control system by delivering the medicament at an increased rate. A suitable equation for adjusting the baseline basal delivery rate is:

$${b}^{′} ( k ) = {P}_{adj}^{0.5} b ( k ) \qquad ( Equation 8 )$$

where b(k) is the baseline basal delivery rate and b′(k) is the adjusted baseline basal delivery rate.

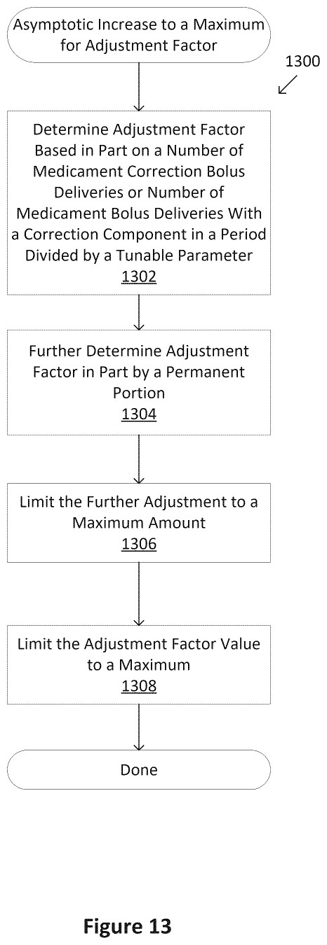

In some exemplary embodiments, the adjustment factor may be calculated to be asymptotically increased to a fixed value to attempt to minimize the number of correction boluses utilized by the user 108. This may be accomplished in part by only making positive increases to the adjustment factor and capping the adjustment factor at a maximum value. Further a portion of adjustment factor may include a permanent portion that is based on a predecessor value. FIG. 13 depicts a flowchart 1300 of illustrative steps that may be performed in exemplary embodiments to adjust the adjustment factor to be asymptotically increased to a fixed value. In this approach, at 1302, the adjustment factor may be determined based in part on the number of medicament correction bolus deliveries over a period or on the number of medicament bolus deliveries with a correction component over the period. The sum may be divided by a tunable value to ensure that the contribution to the adjustment factor does not exceed a maximum. At 1304, the adjustment factor value may be further determined in part by a permanent portion. The permanent portion may be based on a previously determined permanent portion and an adjustment that may be based on most a trend of the most recently determined adjustment factor values for preceding periods. The adjustment may provide for the asymptotic nature of the increase. At 1306, this permanent portion may be limited to a maximum amount. At 1308, the resulting adjustment factor value may be limited to a maximum value, such as 1308.

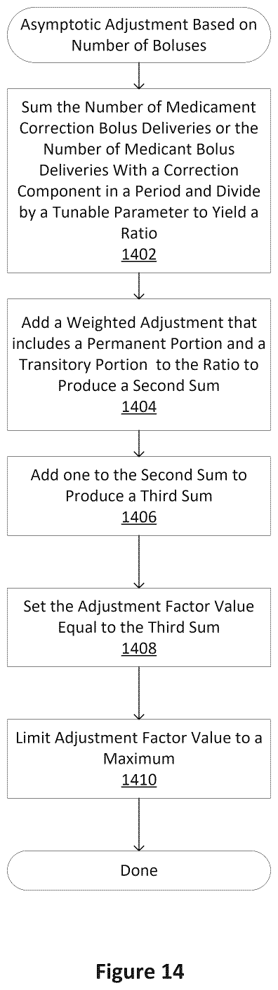

FIG. 14 depicts a flowchart 1400 of illustrative steps that may be performed in exemplary embodiments for a particular approach to providing an adjustment factor that is asymptotically increased to a fixed value to reduce the number of correction boluses or number of boluses with a correction component. A suitable illustrative equation for calculating the adjustment value in this fashion is:

$${P}_{adj , M} ( k ) = min ( 1 + 0.5 {P}_{p , M - 1} + \frac{{Σ}_{j = 1}^{288} {N}_{c o r r} ( j )}{20} , 1.4 ) \qquad ( Equation 9 )$$

where P_{adj,M}(k) is the adjustment factor for period k, P_{p,M-1} is the permanent portion, min( ) is a function that chooses a minimum value, and N_{corr}(j) is the number of medicament correction bolus deliveries and/or a number of medicament bolus deliveries with a correction component occurred in cycle j. It should be appreciated that other equations may be used.

At 1402, the number of medicant correction bolus deliveries over a period or the number of medicament bolus deliveries having a correction component over the period are summed

$$( e . g . {Σ}_{j = I}^{288} {N}_{c o r r} ( j ) ) .$$

The sum may be capped so as to not exceed a value, such as 8. The value 8 may represent a maximum typical number of boluses per period. Otherwise, the contribution of the sum to the adjustment factor value is negated because a minimum value of 1.4 is chosen by the min( ) function since the sum would be greater than 0.4 and added to 1 (as shown in equation 9 and discussed below). The sum may be divided by twenty (a tunable parameter) to yield a ratio. At 1404, a permanent portion may be added to the ratio. The permanent portion includes a weight (e.g., 0.5 in equation 9). The permanent portion is calculated as the most recently calculated permanent portion from a most recent period and an adjustment amount. The adjustment amount may depend on most recent adjustment factor values and the trend in such adjustment factor values. The weighted adjustment is represented by 0.5P_{p,M-1} in equation 9.

A suitable illustrative equation for determining the adjustment amount is:

$${P}_{p , M} = max ( min ( 0.4 , {P}_{p , M - 1} + {P}_{ptrigger , M - 1} ) , 0 ) \qquad ( Equation 10 )$$

wherein P_{p,M} is the adjustment that is weighted in Equation 9, P_{p,M-1} is the adjustment calculated for the preceding period, and P_{ptrigger,M-1} is the adjustment amount. The adjustment amount may be calculated, for example, as:

$${P}_{ptrigger , M - 1} = \{ \begin{matrix}0.05 \\ 1\end{matrix} \frac{\overline{{P}_{adj , M - 1}} > \overline{{P}_{adj , M - 2}} + 0.02}{\overline{\overline{{P}_{adj , M - 1}} \leq \overline{{P}_{adj , M - 2}}} + 0.02} \qquad ( Equation 11 )$$

where P_{p,M-1} is the adjustment factor for period M−1, and P_{p,M-2} is the adjustment factor for period M−2. Hence, the adjustment is less if the most recent adjustment factor is greater than its predecessor.

At 1406, one may be added to the second sum to produce a third sum. At 1408, the adjustment factor may be set as the value of the third sum. At 1410, the adjustment factor may be limited to a maximum value, such as 1.3, 1.4, etc.

While exemplary embodiments have been described herein, it should be appreciated that various changes in form and detail may be made without departing from the intended scope of the claims appended hereto and equivalents thereof.

1. A medicament delivery system for delivering medicament to a user, comprising:
a non-transitory processor-readable storage medium storing programming instructions; and
a processor configured to execute the programming instructions to cause the processor to:
determine a number of boluses of medicament that have been delivered to the user over a period;
determine an adjustment factor based on the determined number of boluses;
adjust at least one glucose control parameter used by the medicament delivery system to control delivery of medicament to the user with the adjustment factor, wherein the adjusting adjusts a rate of medicament delivery to the user by the medicament delivery system.

2. The medicament delivery system of claim 1, wherein the at least one glucose control parameter includes at least one of a target glucose level value, a weight coefficient for a glucose cost component of a cost function, a basal delivery rate to the user, and/or a constraint that specifies a maximum delivery amount of medicament for a specified timeframe.

3. The medicament delivery system of claim 2, wherein the adjusting of the at least one glucose control parameter comprises at least one of decreasing the target glucose level, increasing the weight coefficient for the glucose cost component of the cost function, increasing the basal delivery rate to the user, and/or relaxing the constraint that specified the maximum delivery amount for the specified timeframe.

4. The medicament delivery system of claim 1, wherein the adjusting results in an increase in the rate of medicament delivery.

5. The medicament delivery system of claim 1, wherein the determining of the number of boluses of the medicament that have been delivered to the user over the period determines only a number of correction boluses of the medicament that have been delivered to the user over the period.

6. The medicament delivery system of claim 1, wherein the determining of the number of boluses of the medicament that have been delivered to the user over the period counts a number of correction boluses of the medicament that have been delivered to the user over the period and a number of boluses of the medicament that are not purely correction boluses but that include a correction component that have been delivered to the user over the period.

7. The medicament delivery system of claim 1, wherein the medicament is one that manages or affects a glucose level of the user.

8. The medicament delivery system of claim 1, wherein the adjusting of the at least one glucose control parameter comprises adjusting multiple of the glucose control parameters.

9. A medicament delivery system for delivering medicament to a user, comprising:
a non-transitory processor-readable storage medium storing programming instructions; and
a processor configured to execute the programming instructions to cause the processor to:
determine a sum of bolus doses of medicament that have been delivered to the user over a period;
determine an adjustment factor based on the determined sum of the bolus doses;
adjust at least one glucose control parameter used by the medicament delivery system to control delivery of medicament to the user with the adjustment factor, wherein the adjusting adjusts a rate of insulin delivery to the user by the medicament delivery system.

10. The medicament delivery system of claim 9, wherein the at least one glucose control parameter includes at least one of a target glucose level value, a weight coefficient for a glucose cost component of a cost function, a basal delivery rate to the user, and/or a constraint that specifies a maximum delivery amount of medicament for a specified timeframe.

11. The medicament delivery system of claim 10, wherein the adjusting of the at least one glucose control parameter comprises at least one of decreasing the target glucose level, increasing the weight coefficient for the glucose cost component of the cost function, increasing the basal delivery rate to the user, and/or relaxing the constraint that specified the maximum delivery amount for the specified timeframe.

12. The medicament delivery system of claim 9, wherein the adjusting results in an increase in the rate of medicament delivery.

13. The medicament delivery system of claim 9, wherein the determining of the number of boluses of the medicament that have been delivered to the user over the period determines only a number of correction boluses of the medicament that have been delivered to the user over the period.

14. The medicament delivery system of claim 9, wherein the determining of the sum of bolus doses of medicament that have been delivered to the user over the period determines doses of correction boluses of the medicament that have been delivered to the user over the period and determines portions of the boluses of the medicament which are not purely correction boluses that are for correcting glucose level of the user that have been delivered to the user over the period.

15. The medicament delivery system of claim 9, wherein the medicament is one that manages or affects a glucose level of the user.

16. The medicament delivery system of claim 9, wherein the adjusting of the at least one glucose control parameter comprises adjusting multiple of the glucose control parameters.

17. A medicament delivery system for delivering medicament to a user, comprising:
a non-transitory processor-readable storage medium storing programming instructions; and
a processor configured to execute the programming instructions to cause the processor to:
determine a number of boluses of medicament that have been delivered to the user over a period;
determine an adjustment factor based on the determined number of boluses and upon at least one recent adjustment factor value;
adjust at least one glucose control parameter used by the medicament delivery system to control delivery of medicament to the user with the adjustment factor, wherein the adjusting adjusts a rate of insulin delivery to the user by the medicament delivery system.

18. The medicament delivery system of claim 17, wherein the adjustment factor is determined based on multiple recent adjustment factor values.

19. The medicament delivery system of claim 17, where the determining of the adjustment factor is based in part on whether a most recent adjustment factor value is greater than a second most recent adjustment factor value.

20. The medicament delivery system of claim 17, wherein the determining the adjustment factor comprises determining the adjustment factor so that it does not exceed a maximum value.
